(19) United States
(12) Patent Application Publication
(10) Pub. No.: US 2013/0288354 A1
(43) Pub. Date: Oct. 31, 2013

(54) BIOVESSELS

(71) Applicant: FATE THERAPEUTICS, INC., San Diego, CA (US)

(72) Inventors: Pratik MULTANI, San Diego, CA (US); Paul GRAYSON, La Jolla, CA (US); Daniel SHOEMAKER, San Diego, CA (US)

(21) Appl. No.: 13/929,497

(22) Filed: Jun. 27, 2013

Related U.S. Application Data
(63) Continuation of application No. 13/697,300, now abandoned.
(60) Provisional application No. 61/332,899, filed on May 10, 2010.

(51) Int. Cl. C12M 1/34

(52) U.S. Cl. CPC C12M 41/12
USPC 435/288.1; 435/288.7

(57) ABSTRACT

Provided are improved vessels for cell culture, cell storage, treatment of cells, and cell therapy. The improved vessels comprise one or more devices temperature indicating devices, elapsed time indicating devices and/or devices that indicate environmental conditions of the vessel contents, such as pH, oxygen and carbon dioxide concentration, and glucose concentration.

### BACKGROUND

1. Technical Field

The present invention relates to improved vessels for cell culture, cell storage, and cell therapy. These vessels comprise one or more devices that indicate temperature, time, and/or other environmental conditions to improve the preparation of cells for cell therapy.

2. Description of the Related Art

Cell therapy is becoming a mainstream component of 21^{st} century medical practice. As such there is a need for materials that facilitate the use of cell-based therapeutics. Specifically, the art is in need of improved vessels suitable for use in cell culture, cell, storage, and cell therapy. At present, these complex manipulations cannot be accomplished using a single vessel. Thus, many cell therapy methods described in the art are inefficient and prone to contamination because the cells must be extensively handled before administration to the patient. At present, the art has failed to provide a multi-purpose vessel sufficient to accomplish the manipulation of cells at various temperatures for particular lengths of time in a reproducible and precise fashion. Accordingly, the art is in need of a multi-purpose vessel that can be used for a variety of manipulations, including cell culture, cell storage, cell treatment, and cell therapy.

The vessels of the present invention address these and other related needs in the art.

### BRIEF SUMMARY

The present invention relates to vessels comprising one or more time, temperature, and environmental indicators or combinations thereof.

In one embodiment, the present invention contemplates an endotoxin-free vessel comprising a temperature indicating device comprising at least one temperature indicator that produces a signal that indicates the temperature of the vessel; an elapsed time indicating device that comprises at least one elapsed time indicator; and a population of cells, wherein the vessel is suitable for cell culture, cell storage, cell infusion, treatment of cells, or cell therapy.

In a particular embodiment, the vessel comprises at least one input port and at least one output port for introducing and/or removing substances from the vessel. In certain embodiments, the at least one input port and at least one output port each independently comprises a valve, a filter, or a combination thereof. In some embodiments, the input and output ports are resealable.

In certain embodiments, the at least one temperature indicator is reversible.

In additional related embodiments, the at least one temperature indicator produces a signal that indicates that the temperature of the vessel is above, below, or within a target temperature range.

In a particular embodiment, the at least one temperature indicator produces a signal that comprises a color that corresponds to the temperature of the vessel that is above, below, or within a target temperature range.

In another particular embodiment, the at least one temperature indicator produces a signal that is a digital indication of the temperature of the vessel.

In further embodiments, the at least one temperature indicator comprises a temperature scale that indicates a temperature of the vessel, wherein the temperature indicated is in the range 0° C. to 65° C.

In a related embodiment, the temperature indicated is in the range of 0° C. to 50° C. In another related embodiment, the temperature indicated is in the range of 4° C. to 37° C.

In certain particular embodiment, the at least one elapsed time indicator produces a signal that indicates the elapsed time.

In one embodiment, the at least one elapsed time indicator is activated by a user or by the temperature of the vessel reaching a predetermined threshold temperature.

In another embodiment, the activated elapsed time signal indicates that the vessel has been exposed to a predetermined temperature range for a predetermined elapsed time.

In one embodiment, the elapsed time of any of the preceding embodiments is at least 10 minutes. In one embodiment, the elapsed time of any of the preceding embodiments is at least 1 hour. In one embodiment, the elapsed time of any of the preceding embodiments is at least 2 hours. In one embodiment, the elapsed time of any of the preceding embodiments is at least 4 hours.

In various embodiments, the elapsed time signal that indicates the elapsed time is selected from the group consisting of: a visual signal, an audible signal, an infrared signal, a radio signal, a digital signal, and an analog signal.

In particular embodiments, the elapsed time signal is indicated at predetermined intervals.

In additional embodiments, the elapsed time signal is indicated continuously.

In one embodiment, the vessel comprises an environmental condition indicating device comprising at least one environmental condition indicator that indicates an environmental condition in the vessel selected from the group consisting of: pH, oxygen concentration, carbon dioxide concentration, osmolarity, and glucose concentration.

In a related embodiment, the at least one environmental condition indicator produces a signal if the indicated environmental condition in the vessel is outside a predetermined concentration range.

In particular embodiments, the vessel is selected from the group consisting of: a bag, a pouch, a flask, a three dimensional matrix, and a bioreactor.

In certain embodiments, the bag comprises one or more materials selected from the group consisting of: diethylhexyl phthalate, polyvinylchloride, polyethylene, polypropylene, and fluorinated ethylene propylene.

In a further embodiment, the bag is suitable for centrifugation.

In various embodiments, the volume of the vessel is between 10 mL and 2000 mL. In other various embodiments, the vessel is selected from the group consisting of: 100 mL, 250 mL, 500 mL, 1000 mL, and 2000 mL.

In one particular embodiment, the population of cells is selected from the group consisting of: stem cells, induced pluripotent stem cells, and progenitor cells. In certain particular embodiments, the population of cells is obtained from peripheral blood, umbilical cord blood, bone marrow, or placenta.

In other embodiments, the population of cells comprises hematopoietic stem and/or progenitor cells.

Additional embodiments comprise a cell culture medium.

In particular embodiments, the cell culture medium is a cell growth medium.

In other particular embodiments, the cell culture medium is serum free.

In a further embodiment, the cell culture medium is replaced at regular intervals with another cell culture medium.

In an additional embodiment, the cell culture medium is replaced with a cell storage medium.

In a preferred embodiment, the cell culture medium is replaced with a pharmaceutically acceptable cell culture medium.

In some embodiments, the cell culture medium comprises one or more pharmaceutical agents.

In particular embodiments, the one or more pharmaceutical agents are selected from the group consisting of: a PTH/PTHrP polypeptide, a prostaglandin receptor 2 agonist, a selective PGE2 EP4 agonist, a Wnt pathway agonist, a Hedgehog pathway agonist, and a Notch pathway agonist.

In a preferred embodiment, the vessel comprises a population of hematopoietic stem cells and a pharmaceutical agent, wherein the pharmaceutical agent comprises 16,16 dimethyl PGE2 supplied in dimethyl sulfoxide (DMSO).

In another preferred embodiment, the DMSO is substantially free of methyl acetate, and the 16,16 dimethyl PGE2 is used at a final concentration of about 1 μM to about 100 μM.

In a particular preferred embodiment, the DMSO is substantially free of methyl acetate, and the 16,16 dimethyl PGE2 is used at a final concentration of about 1 μM to about 10 μM.

In another preferred embodiment, the DMSO is substantially free of methyl acetate, and the 16,16 dimethyl PGE2 is used at a final concentration of about 10 μM to about 100 μM.

In one preferred embodiment, the DMSO is substantially free of methyl acetate, and the 16,16 dimethyl PGE2 is used at a final concentration of about 100 μM.

In a certain preferred embodiment, the DMSO is substantially free of methyl acetate, and the 16,16 dimethyl PGE2 is used at a final concentration of about 10 μM.

In another preferred embodiment, the DMSO is substantially free of methyl acetate, and the 16,16 dimethyl PGE2 is used at a final concentration of about 1 μM.

In one embodiment, the present invention contemplates, in part, a kit comprising an endotoxin-free vessel comprising a temperature indicating device comprising at least one temperature indicator that produces a signal that indicates the temperature of the vessel; an elapsed time indicating device that comprises at least one elapsed time indicator; and a population of cells, one or more pharmaceutical agents; and instructions for using the kit.

In one particular embodiment, the kit comprises a population of cells selected from the group consisting of: stem cells, induced pluripotent stem cells, and progenitor cells. In certain particular embodiments, the population of cells is obtained from peripheral blood, umbilical cord blood, bone marrow, or placenta.

In other embodiments, the kit comprises hematopoietic stem and/or progenitor cells.

In additional embodiments the kit comprises a cell culture medium.

In particular embodiments, the kit comprises a cell culture medium that is a cell growth medium.

In other particular embodiments of the kit, the cell culture medium is serum free.

In a further embodiment of the kit, the cell culture medium is replaced at regular intervals with another cell culture medium.

In an additional embodiment of the kit, the cell culture medium is replaced with a cell storage medium.

In a preferred embodiment of the kit, the cell culture medium is replaced with a pharmaceutically acceptable cell culture medium.

In a certain embodiment of the kit, the one or more pharmaceutical agents are selected from the group consisting of: a PTH/PTHrP polypeptide, a prostaglandin receptor 2 agonist, a Wnt pathway agonist, a Hedgehog pathway agonist, and a Notch pathway agonist.

In a preferred embodiment of the kit, the pharmaceutical agent is supplied in a lyophilized form in an endotoxin-free vessel.

In another preferred embodiment of the kit, the population of cells comprises hematopoietic stem cells, and the pharmaceutical agent comprises 16,16 dimethyl PGE2 supplied in dimethyl sulfoxide (DMSO).

In a certain preferred embodiment of the kit, the DMSO is substantially free of methyl acetate, and the 16,16 dimethyl PGE2 in DMSO is supplied in a 2 ml vial with a Teflon coated stopper, wherein there is an air overlay in the vial.

In a particular preferred embodiment, a kit comprises PGE2 in

DMSO at a concentration of PGE2 of 10 mM, 100 mM, or 1 M. In one embodiment, a kit comprises PGE2 in DMSO at a concentration of about 10 mM.

In one embodiment, the present invention provides a method of preparing a population of cells to administer to a subject comprising selecting a subject in need of cell therapy; providing a population of cells to be cultured; culturing the population of cells in an endotoxin-free vessel comprising a temperature indicating device comprising at least one temperature indicator that produces a signal that indicates the temperature of the vessel, and an elapsed time indicating device that comprises at least one elapsed time indicator, and a population of cells; contacting the population of cells with one or more pharmaceutical agents at a temperature and for a duration sufficient to prepare the cells for administering to a subject; and administering the prepared population of cells to the subject.

In a particular embodiment, the patient in need of therapy is a bone marrow donor who has donated bone marrow, is a bone marrow donor who has yet to donate bone marrow, is a bone marrow donor transplant recipient, has hematopoietic progenitor cells under environmental stress, has anemia, has a reduced level of immune cell function compared to a normal subject, or has an immune system deficiency.

In a certain embodiment, the patient in need of therapy has myeloma, non-Hodgkin's lymphoma, Hodgkin's lymphoma, chronic myeloid leukemia, chronic myelogenous leukemia, chronic granulocytic leukemia, acute lymphoblastic leukemia, acute nonlymphoblastic leukemia, or pre-leukemia.

In an additional embodiment of the method of the invention, the population of cells comprises hematopoietic stem cells, and the pharmaceutical agent comprises 16,16 dimethyl PGE2 supplied in dimethyl sulfoxide (DMSO). In a particular preferred embodiment, the method of the invention comprises PGE2 in DMSO at a concentration of 10 mM, 100 mM, or 1 M. In one embodiment, the method comprises PGE2 in DMSO at a concentration of about 10 mM.

In a further embodiment of the method, the DMSO is substantially free of methyl acetate, and the 16,16 dimethyl PGE2 is added to a final concentration of about 1 μM to about 100 μM. In a particular preferred embodiment, the DMSO is substantially free of methyl acetate, and the 16,16 dimethyl PGE2 is used at a final concentration of about 1 μM to about 10 μM. In another preferred embodiment of the method, the DMSO is substantially free of methyl acetate, and the 16,16 dimethyl PGE2 is used at a final concentration of about 10 μM to about 100 μM. In another preferred embodiment, the DMSO is substantially free of methyl acetate, and the 16,16 dimethyl PGE2 is used at a final concentration of about 10 μM.

In a particular embodiment of the method, the population of cells is contacted with one or more therapeutic agents for about 10 minutes.

In another particular embodiment of the method, the population of cells is contacted with one or more therapeutic agents for about 1 hour.

In another particular embodiment of the method, the population of cells is contacted with one or more therapeutic agents for about 2 hours.

In another particular embodiment, the population of cells is contacted with one or more therapeutic agents for about 4 hours.

In certain embodiments of the method, the cell growth medium is replaced after contacting the population of cells with one or more pharmaceutical agents for the duration sufficient to prepare the cells for administering to a subject.

In other embodiments of the method, the population of cells in the vessel is transferred to a new vessel prior to administration to the subject.

In a preferred embodiment of the method, the cells are parenterally administered.

In various embodiments, the parenteral administration is selected from the group consisting of: intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In any one or more of the preceding embodiments, the invention contemplates, in part, the use of a vessel as disclosed herein, wherein the vessel is or is a component of, or is used in combination with, a closed cell processing system.

In any one or more of the preceding embodiments, the invention contemplates, in part, a vessel that forms or is a component of a closed cell processing system. In some embodiments, the vessel is, or is a component of, a closed cell processing system that automates one or more cell processing steps.

In a particular embodiment, the one or more cell processing steps automated by the closed cell processing system is selected from the groups consisting of: cell incubation, cell culture, cell treatment, cell storage, centrifugation, cell selection, media addition, media exchange, cell washing, and cell infusion.

### DETAILED DESCRIPTION

As used in this specification and the appended claims, the singular forms “a,” “an” and “the” include plural references unless the content clearly dictates otherwise.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

It is to be understood that the terms “top”, “bottom”, “upper”, “lower”, “right”, “left”, “vertical”, “horizontal”, “length”, “width”, “height”, “thickness”, “front”, “back”, “rear”, “side” and the like are used herein merely to describe points of reference and do not limit the present invention to any specific configuration or orientation.

### A. Vessels

The invention provides vessels for manipulating cells in a precise manner using a single vessel. To improve cell therapy techniques, the vessels of the invention monitor environmental conditions, including temperature, time, and/or pH, to allow cell culture, cell storage, and cell treatment under desired conditions in a reproducible and precise manner. The contemplated vessels have devices for indicating temperature and/or environmental conditions of the vessels, allowing precise control over incubation conditions for incubating a population of cells with an agent, including incubation temperature and time.

The invention is based, in part, on vessels for precisely manipulating cells useful in cell therapy comprising one or more indicators that indicate, for example, the temperature of the vessel contents, the time the vessel has been at any given temperature, and various environmental conditions. As used herein, the term “vessel” relates generally to any container capable of being used for purposes of culturing, handling, manipulating, storing, analyzing, incubating, administering and otherwise establishing, supporting, growing, harvesting, treating, and using cells and by-products thereof ex vivo or in vitro or otherwise for a variety of purposes as set forth and as contemplated herein.

In various embodiments, vessels of the invention form a closed processing system, or in other embodiments vessels of the invention are used in combination with, or are a component of, a closed cell processing system. Closed cell processing systems automate processes including treatment, centrifugation, incubation, media addition, cell selection, cell washing, and final fill and finish within “closed” or resealable vessels of the invention. Closed cell processing systems integrate and automate the processes and replicate many qualitatively controlled manual tasks to provide consistent and operator-independent quality.

Benefits delivered from an automated, closed-cell processing system would include significant reduction in the cost of therapies (typically 25-90%) and the number of operators required (typically >70%); lowered dependence on skilled labor; significant savings in capital investment through better facility use (typically 30-50%); improved quality and fewer quality events; and an ability to more rapidly scale up and scale out to match market demands.

Exemplary closed cell processing systems useful in the invention, include, but are not limited to CaridianBCT's Quantum Cell Expansion System and Elutra System, Miltenyi Biotec's CliniMACs Prodigy System, BioSafe SA's Sepax System, KBI Biopharma's kSep System, and those systems disclosed in, for example, International Patent Application Publication Numbers WO2010/008563 and WO2010/008579.

Types

Illustrative embodiments of vessels include, but are not limited to: bags (e.g., intravenous (IV) bags; cell culture bags, e.g., VueLife™ KryoSure™, KryoVue™, Lifecell®, PermaLife™, X-Fold™, Si-Culture™ VectraCell™), bioreactors, cell or tissue culture devices, pouches, capsules, culture vials, apparatuses, cell factories, containers, culture tubes (e.g., microcentrifuge tubes, EPPENDORF TUBES®, FALCON® conical tubes, etc.), culture dishes (e.g., Petri dishes), culture flasks, spinner flasks, roller bottles, multi-well plates (e.g., 2-well, 4-well, 6-well, 12-well, 24-well, 48 well, 96-well, and 384-well plates), micro-incubators, micro-carriers, microplates, microslide and chamber slides, and implant devices (e.g., collagen sponges). The vessel may be used multiple times or may be a single-use vessel.

The vessel of the invention may be open, such as a culture flask, or may be a closed system such that the contents of the vessel are not open to the outside environment. The vessel may be a closed cell processing system that automates one or more cell manipulation steps, such as treating, washing, culturing, and incubating cells. In some embodiments, the vessel is used in combination with, or forms a component of, a closed cell processing system.

Endotoxin Free

Preferably, vessels of the present invention are endotoxin free, and manufactured according to GMP practices. As used herein, the term “endotoxin free” refers to vessels and/or compositions that contain at most trace amounts (i.e., amounts having no adverse physiological effects to a subject) of endotoxin, and preferably undetectable amounts of endotoxin. In one embodiment, the term “endotoxin free” refers to a vessel and/or compositions that is at least 95%, at least 96%, at least 97%, at least 98%, at least 99%, or 100% endotoxin free. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipooligosaccharides (LOS) found in the outer membrane of various Gram-negative bacteria, and which represent a central pathogenic feature in the ability of these bacteria to cause disease. Small amounts of endotoxin in humans can produce fever, a lowering of the blood pressure, and activation of inflammation and coagulation, among other adverse physiological effects. Therefore, it is often desirable to remove most or all traces of endotoxin from drug product containers, because even small amounts may cause adverse effects in humans. Endotoxins can be removed from vessels using methods known in the art, for example, vessels can be cleaned in HEPA filtered washing equipment with endotoxin-free water, depyrogenated at 250° C., and clean-packaged in HEPA filtered workstations located inside a class 100/10 clean room (e.g., a class 100 clean room, contains no more than 100 particles bigger than half a micron in a cubic foot of air).

GMP

As used herein, the term “good manufacturing practice (GMP)” refers to the control and management of manufacturing, and quality control testing, of foods, pharmaceutical products, and medical devices. GMP does not necessarily rely on sampling, but instead relies on documentation of every aspect of the process, activities, and operations involved with drug and medical device manufacture. If the documentation showing how the product was made and tested (which enables traceability and, in the event of future problems, recall from the market) is not correct and in order, then the product does not meet the required specification and is considered contaminated (i.e., adulterated in the US). Additionally, GMP typically requires that all manufacturing and testing equipment has been qualified as suitable for use, and that all operational methodologies and procedures (e.g., manufacturing, cleaning, and analytical testing) utilized in the drug manufacturing process have been validated according to predetermined specifications to demonstrate that they can perform their purported function(s). In the US, the phrase “current good manufacturing practice” appears in 501(B) of the 1938 Food, Drug, and Cosmetic Act (21 U.S.C. §351).

Materials

In particular embodiments, vessels can be fabricated from materials that comprise one or more of the following characteristics: gas permeability (materials have suitable gas transfer rates for oxygen, carbon dioxide and nitrogen); negligible water loss rates (materials are practically impermeable to water); chemically and biologically inert (materials do not react with the vessel contents), and retention of flexibility and strength in various conditions (materials enable vessel to be microwaved, treated with UV irradiation, centrifuged, or used within a broad range of temperatures, e.g., from −100° C. to +100° C.).

Those skilled in the relevant art can select appropriate polymeric materials with the desired permeability, bioreactive and biocompatible properties, temperature resistance, flexibility, heat conductivity, and strength.

In various illustrative embodiments, it may be desirable to minimize leaching of the material into the contents of the vessel (e.g., culture media). In particular embodiments, in which cultured cells or their products may be used in humans, plants, animals, or other organisms, it is further desirable that the polymeric materials have little or no detrimental effect on the human, plant, animal, microbe, or the like. More preferably, the Food and Drug Administration will have designated the material as safe in Title 21 of the United States Code of Federal Regulations (CFR) Parts 170 through 199 and parts 800 through 1299.

In related embodiments, it may be desirable to fabricate vessels of the invention using materials that are at least somewhat resistant to acids, bases, salts, or other potentially harsh or corrosive reagents, media, and buffers.

In other illustrative embodiments, it may be desirable to fabricate vessels from materials that are tolerant to incubation and/or storage at a wide range of temperatures, transportation and handling, centrifugation, repeated freeze-thaw cycles, irradiation, high temperature incubation, low-temperature and cryogenic storage, autoclave sterilization, and/or combinations thereof as well as other molecular biology, clinical, industrial, or research applications.

In another particular embodiment, a vessel is fabricated from materials that conduct heat, i.e., the temperature the surrounding environment (e.g., a preset incubator, an ice bath) is transferred readily to the vessel contents. Thus, the temperature of the vessels contents, the vessel, and the surrounding environment are substantially the same.

Exemplary materials that are suitable for fabricating vessels of the present invention include, but are not limited to: glass, ceramics, metals, thermoset and elastomer monomers and polymers, and monomeric and polymeric thermoplastics. Exemplary thermoplastic materials suitable for fabricating vessels of the present invention include, without limitation: acetal resins, delrin, fluorocarbons, polyesters, polyester elastomers, metallocenes, polyamides, nylon, polyvinyl chloride, polybutadienes, silicone resins, ABS (an acronym for “acrylonitrile, butadiene, styrene”), polycarbonate (also referred to in the plastics industry as “PC”) polypropylene, polyethylene, polystyrene, liquid crystal polymers, alloys and combinations and mixtures and composites thereof, and reinforced alloys and combinations and mixtures and composites thereof.

The amenability of the material to roller manufacture, injection molding manufacture, or other preferred or possible methods of manufacture may also influence the choice of materials used to fabricate vessels contemplated by the present invention.

In addition to these and other criterion that may be somewhat specific to molecular biology or cell culture, additional factors such as price, availability, durability, resistance to scuffing or impact, and the like may also influence the appropriate choice of materials used to fabricate vessels contemplated by the present invention.

In particular embodiments, vessels can be fabricated from one or more materials selected from the group consisting of: diethylhexyl phthalate, polyvinylchloride, polyethylene, polypropylene, and fluorinated ethylene propylene.

In a certain embodiment, a vessel is fabricated from fluorinated ethylene propylene.

Dimensions

In various illustrative embodiments, a vessel can be fabricated to have a cross-sectional wall thickness that is based upon and that is an implicit function of the selected materials and the intended applications. Exemplary cross-sectional wall thicknesses include, without limitation, thicknesses between about 0.25 mm and about 2.0 mm, between about 0.5 mm and about 1.5 mm, and between about 0.75 mm and about 1.25 mm In particular embodiments, the cross-sectional wall thickness of a vessels is at least 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm or any intervening thickness.

In particular illustrative embodiments, vessels of the present invention are designed to accommodate specific volumes. Exemplary volumes of the vessels of the present invention include, without limitation, volumes of about 10 mL, about 25 mL, about 50 mL, about 75 mL, about 100 mL, about 150 mL, about 250 mL, about 500 mL, about 750 mL, about 1000 mL, about 1250 mL, about 1500 mL, about 1750 mL, about 2000 mL, or more, including any intervening volume. For example, intervening volumes between 10 mL and 25 mL, include 11 mL, 12 mL, 13 mL, 14 mL, 15 mL, 16 mL, 17 mL, 18 mL, 19 mL, 20 mL, 21 mL, 22 mL, 23 mL, and 24 mL.

In certain embodiments, a vessel contemplated herein comprises 1, 2, 3, 4, or 5 compartments. The compartments can be the same size or different sizes and may have the same or different porosities. In one embodiment, the porosity of adjacent compartments is such that small molecules, nutrients, polypeptides, and/or growth factors may freely be exchanged between compartments, but wherein the cells of each compartment are restricted to their respective compartments. Such designs are useful when the production of a cellular factor is required for a desired effect on an adjacent compartment of cells.

One having skill in the relevant art can fabricate a vessel having the desired thickness, volume, and number of compartments based on knowledge of the fabrication materials and the intended uses of the vessel.

Coatings

In particular embodiments, vessels can be fabricated from materials that accommodate particular coatings, films, or other agents, and in some embodiments the internal surfaces of the vessel may be coated with particles such as, for example, reagents, chemical activators or inhibitors, or drugs. For example, in some embodiments the interior surface of the vessel is coated with a particular reagent, such as a drug substance, to allow exposing or treating cells introduced into the vessel with the reagent. In some embodiments, the interior surface of the vessel is coated with a drug such as a PTH/PTHrP polypeptide; a prostaglandin receptor agonist (e.g., PGE2 and PGE4 receptor agonists); a Wnt pathway agonist (e.g., Wnt agonists, GSK-3β antagonists); a Hedgehog pathway agonist; or a Notch pathway agonist, as discussed elsewhere herein.

The interior surface of the vessel can be designed to accommodate coating with various hydrophobic, hydrophilic, or amphipathic molecules using methods known to those in the relevant art. For example, commonly used hydrophobic materials such as for example, thermoset materials, elastomers, rubbers, and thermoplastics such as polystyrenes, polycarbonates, ABSs, and other polymeric materials disclosed herein accommodate binding of hydrophobic molecules (e.g., proteins having one or more hydrophobic regions). Furthermore, specific experimental, industrial, or clinical applications may require, preclude, or be indifferent to the binding of various types of molecules such as, for example, nucleic acids, proteins, carbohydrates, or the like. It may also be desirable to prevent, minimize, and or maximize binding molecules to the substrate depending upon the objectives of a particular application.

Ports

In particular illustrative embodiments, the invention contemplates vessels comprising one or more ports, which are preferably resealable. The ports can be input and/or output ports for introducing, exchanging, or removing compounds, cells, cell culture medium, and other substances from the vessel. The ports can provide needle-less access or can include access points for needles, as appropriate. Each port may contain one or more adapters (e.g., luer adapters), valves and/or filters. In various embodiments, the ports are designed to be suitable for use with open, closed, or open and closed cell processing systems.

Filters may be important in excluding or removing most microbes such as bacteria and fungi, larger cells, debris, and other possible contaminants from the vessel. Exemplary filter porosities include, 0.2 micron filters for removal of bacteria, fungi, and other microbes and 10-20 micron filters for removing larger debris. Other exemplary filter sizes can range from about 0.01 microns to about 200 microns depending on the particular applications of the vessel. Filters may be used alone or in combination with other filters. Filters are or may be available as hybrid filters that combine various filter materials or pore sizes. Alternatively, a filter may have a gradient of pore size, from relatively large to relatively small pores,

In addition to pore size, filters are also available in a plurality of materials. Filters can be made of one or a combination of many different materials such as, for example, glass, polypropylene, polyvinyl chloride, polycarbonate, polytetrafluoroethylene, polyvinylidiene fluoride, mixed cellulose esters, polyether sulfone, nylon, or the like. Filters are commercially available from several vendors including Millipore of Bedford, Mass. USA and Porex Corporation of Fairburn, Ga., USA.

### B. Devices

The invention contemplates, in part, vessels comprising one or more devices that indicate, for example, the temperature of the vessel contents, the time the vessel has been at any given temperature, and various environmental conditions (e.g., pH, oxygen concentration, carbon dioxide concentration, glucose concentration). The invention contemplates devices in the form of cards, strips, disks, stickers, labels, probes, sensors, and small electronic devices, and other suitable devices that indicate environmental conditions inside the vessel, such as temperature. The contemplated devices can be integrated into the material of the vessel or alternatively, can be manufactured separately from the vessel and subsequently, permanently or non-permanently affixed or adhered to the vessel.

The device may indicate the temperature or other environmental condition by any manner of signal. Various types of signals are contemplated for use in indicators of the invention. For example, indicators can indicate temperature or environmental condition by producing a visual signal (e.g., a digital display, a color change, a graph), an audible signal, an infrared signal, a radio signal, an analog signal, a digital signal, or combinations thereof. In some embodiments, the signal is displayed on the outer surface of the vessel, and in some embodiments, the signal is transmitted to an electronic device or equipment, such as a computer, tracking device, or cell phone. In other embodiments, the signal is transmitted to and displayed on a closed cell processing system used in combination with a vessel comprising one or more devices as disclosed herein.

In one embodiment, a vessel comprises a temperature indicating device. In a preferred embodiment, a vessel comprises both a temperature indicating device and an elapsed time indicating device, either separately as individual devices or combined as a single device. In further embodiments, the vessel comprises a temperature indicating device, an elapsed time indicating device and one or more devices that indicate an environmental condition. The plurality of devices may be fabricated separately as individual devices or fabricated as a single device.

Temperature Indicating Devices

The present invention contemplates, in part, vessels suitable for cell culture, cell storage, cell infusion, treatment of cells, and/or cell therapy, wherein the vessels comprise at least one temperature indicating device. As used herein, the term “temperature indicating device” refers to a device that senses, measures, and/or indicates a temperature of the vessel and/or vessel contents and that comprises one or a plurality of “temperature indicators.”

In a particular embodiment, the vessel is fabricated from materials that conduct heat, i.e., the temperature of the surrounding environment (e.g., a preset incubator, an ice bath) is transferred readily to the vessel contents. In a preferred embodiment, the vessel is fabricated from materials that conduct heat, such that the temperature of the vessel is substantially the same as the vessel contents. Accordingly, in such embodiments, a temperature indicator indicates the temperature of the vessel contents and the vessel because the two temperatures are substantially the same.

As used herein, the term “temperature indicator” refers to an indicator that produces a signal that indicates a temperature. The temperature indicator can produce a signal that corresponds to the real-time temperature or exposure to a particular temperature for any predetermined length of time.

The temperature indicating device can be fabricated integral with the vessel, can be permanently or non-permanently attached to the vessel exterior surface, can be laminated to the vessel exterior surface or can be encased with the vessel within a vessel liner. The device may be a small electronic device, probe, sensor, or combinations thereof.

The temperature indicating device may be of any size or shape. Exemplary forms of the temperature indicating device, include, but are not limited to: cards, strips, disks, stickers, labels, probes, electronic devices, or combinations thereof. For example, a temperature indicating device in the form of a card or strip may comprise one or a plurality of temperature indicating disks or vice versa.

The temperature indicating device can be user-activated, or activated by exposure to a predetermined temperature. In addition, each temperature indicator in a temperature indicating device that comprises a plurality of temperature indicators can be activated at different predetermined temperatures. As used herein, the term “predetermined temperature” refers to a temperature or temperature range selected for monitoring by the user. In various embodiments, the predetermined temperature is a “target temperature” that indicates the desired temperature or temperature range for which the vessel is contemplated for use. In other embodiments, a predetermined temperature is an over-temperature or under-temperature of the vessel that is outside the contemplated target temperature or temperature range. In various embodiments, a temperature indicating device provides temperature indicators that indicate one or more predetermined temperatures, i.e., temperatures of the vessel and/or vessel contents at, above, or below a target temperature, or above, below or within a target temperature range.

Exemplary target temperatures, including over-temperatures and under-temperatures, include without limitation, 0° C., 4° C., 20° C., 25° C., 37° C., 42° C., 50° C., 65° C., 100° C., or any intervening temperature. One having skill in the art can select the target temperature based on the particular application(s) for the vessel and/or vessels contents. Accordingly, the temperature indicator that indicates the target temperature can be reliably calibrated to any temperature in the range of about −100° C. to about 100° C. using methods known in the relevant art.

In other exemplary embodiments, target temperatures may be the median in a range of temperatures. For example, temperature ranges of about −2° C. to about 6° C., about 18° C. to about 22° C., about 23° C. to about 27° C., about −35° C. to about 39° C., about 40° C. to about 44° C., about 45° C. to about 55° C., about 60° C. to about 70° C., or about 95° C. to about 105° C. The present invention contemplates a variety of target temperatures and temperature ranges, without limitation.

In particular embodiments, a temperature indicating device comprises one or more temperature indicators that reversibly indicate the temperature, i.e., sensing, measuring, and indicating the temperature of a vessel and/or vessel contents in real-time. In other embodiments, the one or more temperature indicators irreversibly indicate that the vessel and/or vessel contents have reached a predetermined temperature or have experienced a predetermined temperature for a particular length of time. In further embodiments, a temperature indicating device comprises one or more reversible and irreversible temperature indicators in any number or combination. The reversible and irreversible temperature indicators can be fabricated in the same temperature indicating device or different temperature indicating devices.

Various types of signals are contemplated for use in temperature indicators of the present invention. For example, temperature indicators can indicate temperature by producing a visual signal (e.g., a digital display, a color change, a graph), an audible signal, an infrared signal, a radio signal, an analog signal, a digital signal, or combinations thereof.

For example, the temperature indicating device can include one or more of: a liquid crystal display (LCD) to indicate temperature; a voice or speech synthesizer to indicate temperature or to specify that an item is below or exceeds a predetermined target temperature; an analog, infrared, or radio signal that indicates the temperature via sound, ultra-sonic or other waves; and/or a digital signal that indicates the temperature electronically.

In particular embodiments, the temperature indicating device comprises one or more temperature indicators that produce a signal that indicates that the temperature of the vessel and/or vessel contents are at, above, or below a target temperature, or above, below or within a target temperature range. A plurality of target temperatures and temperature ranges may be monitored by using a single temperature indicating device comprising a plurality of temperature indicators.

Temperature indicators that produce visual indications of temperature can produce a signal that comprises a color that corresponds to a temperature of the vessel and/or vessel contents that are at, above, or below a target temperature, or above, below or within a target temperature range. In one illustrative embodiment, a temperature indicating device comprises: one or more temperature indicators that can produce a signal comprising a blue color to indicate temperatures below the target temperature or target temperature range; one or more temperature indicators that can produce a signal comprising a green color to indicate temperatures at the target temperature or within the target temperature range; and one or more temperature indicators that can produce a signal comprising a red color to indicate temperatures above the target temperature or target temperature range. The present invention contemplates that any color combinations can be used with any number and combination of temperature indicators, without limitation. One having skill in the art could employ any number of chemicals which reflect certain colors at certain temperatures and could select such chemical substances to reflect a desired color to correspond to a particular temperature. Exemplary temperature sensing substances are disclosed in U.S. Pat. No. 3,651,695 (Brown), U.S. Pat. No. 3,861,213 (Parker), U.S. Pat. No. 3,864,976 (Parker), U.S. Pat. No. 4,859,360 (Suzuki et al) and U.S. Pat. No. 5,806,528 (Magliochetti).

Temperature indicators can also produce a digital signal that indicates the temperature of the vessel and/or vessel contents that are at, above, or below a target temperature, or above, below or within a target temperature range or alternatively, or in addition, produces a digital signal that indicates the precise temperature of the vessel and/or vessel contents. In various embodiments, the digital signal indicates the temperature as an alphanumeric signal produced on an LED, an organic light emitting diode (OLED), or an LCD.

In one embodiment, the temperature indicators comprise a short-range wireless interface, such as a BLUETOOTH interface, ZIGBEE, or WIFI interface, a long range cellular phone or satellite communications interface, or a wired interface, such as a RS 232, USB or FIREWIRE interface.

In additional illustrative embodiments, temperature indicating devices can comprise one or a plurality of temperature scales each comprising one or more temperature indicators that indicate a predetermined temperature or temperature range. Each temperature indicator within a temperature scale can produce a signal (e.g., visual signal, digital signal).

In one illustrative embodiment, each temperature indicator in a temperature scale comprises a corresponding temperature sensitive substance that indicates the temperature, e.g., by changing color or by illuminating a desired temperature range of the temperature scale. In other words, each temperature indicator in a temperature scale is typically associated with a temperature sensitive substance having a particular temperature threshold range corresponding to that temperature or temperature range. When the temperature of the vessel and/or vessel contents are within the threshold temperature range of a substance, that substance changes color or illuminates the associated temperature indicator to produce a signal that indicates the temperature. For example, if the temperature of 37° C. then the temperature indicator representing 37° C. or a range comprising 37° C. on the temperature scale produces a signal to indicate the temperature. It would be understood that the temperature thresholds can be adjusted in any manner to enable one or a plurality of temperature indicators to produce a signal in response to a temperature residing between successive temperature indicators in a temperature scale. Further, the temperature thresholds can be set to produce a signal for a successive indicator (e.g., a 37° C. target temperature indicator may be set to produce a signal at any temperature between 35° C. and 39° C.).

Temperature scales illustratively include temperature indicators in increments of 1° C., 2° C., 3° C., 4° C., 5° C., 6° C., 7° C., 8° C., 9° C., 10° C., over ranges of 5° C., 10° C., 15° C., 20° C., 25° C., 30° C., 35° C., 40° C., 45° C., 50° C., 55° C., 60°0 C., 65° C., 70° C., 75° C., 80° C., 85° C., 90° C., 95° C., 100° C., 110° C., 125° C., 130° C., 140° C., 150° C., 160° C., 170° C., 180° C., 190° C., or 200° C. or more.

Further exemplary temperature ranges include, without limitation, ranges of about −100° C. to about 100° C., about −80° C. to about 100° C., about −20° C. to about 100° C., about −10° C. to about 100° C., about 0° C. to about 100° C., about 4° C. to about 100° C., about −20° C. to about 65° C., about −20° C. to about 50° C., about −10° C. to about 65° C., about 0° C. to about 65° C., about 0° C. to about 50° C., about 0° C. to about 42° C., about 0° C. to about 37° C., about 4° C. to about 65° C., about 4° C. to about 50° C., about 4° C. to about 42° C., about 4° C. to about 37° C. or any intervening ranges of temperatures.

In one non-limiting example, a single temperature scale may indicate a temperature in a range of 0° C. to 65° C. and include 14 temperature indicators in successive 5° C. increments, e.g., 0° C., 5° C., 10° C., 15° C., 20° C., 25° C., 30° C., 35° C., 40° C., 45° C., 50° C., 55° C., 60° C., and 65° C. In one illustrative embodiment, a temperature of 4° C. would produce a signal for both the 0° C. and 5° C. temperature indicators; a temperature of 22° C. would produce a signal for both the 20° C. and 25° C. temperature indicators; and a temperature of 37° C., would produce a signal for both the 35° C. and 40° C. temperature indicators. The present invention contemplates related embodiments having increments of 1° C., 2° C., 3° C., 4° C., 5° C., 6° C., 7° C., 8° C., 9° C., 10° C. over both wider and narrower temperature ranges.

In another illustrative embodiment, a single temperature scale may indicate a temperature in a range of 0° C. to 65° C. and include 14 temperature indicators in successive 5° C. ranges, e.g., 0° C.-5° C., 6° C.-10° C., 11° C.-15° C., 16° C.-20° C., 21° C.-25° C., 26° C.-30° C., 31° C.-35° C. 36° C.-40° C., 41° C.-45° C., 46° C.-50° C., 51° C.-55° C., 56° C.-60° C., and 61° C.-65° C. In one illustrative embodiment, a temperature of 4° C. would produce a signal for temperature indicator that indicates a temperature for the range of 0° C.-5° C.; a temperature of 22° C. would produce a signal for temperature indicator that indicates a temperature for the range of 21° C.-25° C.; and a temperature of 37° C. would produce a signal for temperature indicator that indicates a temperature for the range of 36° C.-40° C. In preferred embodiments, the signal can be a color that illuminates the corresponding temperature indicator or an alphanumeric representation of the precise temperature of the vessel and/or vessel contents on the corresponding temperature indicator. The present invention contemplates related embodiments having increments of 1° C., 2° C., 3° C., 4° C., 5° C., 6° C., 7° C., 8° C., 9° C., 10° C. over both wider and narrower temperature ranges.

The invention contemplates, without limitation, that a temperature indicating device can comprise one or more temperature scales, each scale comprising any number and combination of temperature indicators, in any increments over any temperature range to indicate a predetermined temperature or temperature range.

In addition, a temperature indicating device can comprise one or more reversible or irreversible temperature indicators that indicate when the vessel and/or vessel contents have reached a particular temperature or has experienced a temperature for a determined length of time. Irreversible temperature indicators may be useful to indicate when the vessel contents have experienced a desired or undesired temperature for a particular period of time. In preferred embodiments, the irreversible temperature indicator indicates a visual signal and/or audio signal. The visual signal can be in the form of a permanent color change that indicates a particular temperature has been reached or experienced for a predetermined length of time. The audible signal can be in the form of an alarm that requires manual silencing.

In further embodiments, the temperature indicating device comprises a temperature indicator that produces a warning signal that the vessel temperature is approaching a maximum desired temperature, such as the maximum temperature allowable for preserving the viability of the vessel contents or performing a reaction in the vessel.

In one embodiment, the temperature indicating device comprises a plurality of reversible temperature indicators each associated with a specific temperature range and one or more irreversible temperature indicators that indicate when one or a plurality of predetermined temperatures have been reached or experienced for any predetermined length of time. The reversible indicators individually provide visual indications of the temperature in real time. The visual indications of temperature correspond to the temperature of the vessel and/or vessels contents that are at, above, or below a target temperature, or above, below or within a target temperature range. An irreversible indicator maintains a visual indication once the predetermined temperature has been reached.

The invention further contemplates a vessels comprising one or more temperature indicating devices and one or more elapsed time indicating devices, or devices that indicate various environmental conditions.

Elapsed Time Indicating Devices

The invention contemplates, in part, vessels suitable for cell culture, cell storage, cell infusion, treatment of cells, and/or cell therapy, wherein the vessels comprise at least one elapsed time indicating device. In one embodiment, a vessel comprises at least one temperature indicating device and one elapsed time indicating device. As used herein, the term “elapsed time indicating device” refers to a device comprising one or a plurality of “elapsed time indicators.” As used herein, the term “elapsed time indicator” refers to an indicator that measures, monitors, and indicates when a predetermined length of time has elapsed. Each elapsed time indicator can be independently user-activated or activated by exposure to a particular predetermined temperature or temperature range.

Once activated, an elapsed time indicator indicates the time from activation. In one embodiment, a given elapsed time indicator measures a predetermined amount of time and then produces a signal that indicates when the predetermined amount of time has elapsed. In various illustrative embodiments, an elapsed time indicating device comprises 1, 2, 3, 4, 5, or more elapsed time indicators, each being individually capable of independent activation by a user or by exposure to the same or a different predetermined temperature or temperature range.

In various embodiments, the present invention contemplates, in part, a vessel comprising an elapsed time indicator that indicates the time the vessel and/or contents have been exposed to, experienced, or maintained at one or a plurality of predetermined temperatures or temperature ranges. In a related embodiment, the elapsed time indicator indicates a predetermined amount of time the vessels and/or contents have been exposed to, experienced, or maintained at one or a plurality of predetermined temperatures or temperature ranges. The elapsed time indicator can produce a continuous signal or one or a plurality of signals at points at which a predetermined percentage of the total predetermined time has elapsed, e.g., produces a signal at regular intervals of the total elapsed time to be measured.

The elapsed time indicating device can be fabricated integral with the vessel, can be permanently or non-permanently attached to the vessel exterior surface, can be laminated to the vessel exterior surface or can be encased with the vessel within a vessel liner. The device can be a small electronic device, a probe, a sensor, or a combination thereof.

The elapsed time indicating device may be of any size or shape. Exemplary shapes of the elapsed time indicating device, include, but are not limited to: cards, strips, disks, stickers, labels, probes, or combinations thereof.

For example a temperature indicating device in the form of a card or strip may comprise one or a plurality of temperature indicating disks or vice versa.

In one illustrative embodiment, an elapsed time indicator indicates an elapsed time of about 1 minute, about 5 minutes, about 10 minutes, about 15 minutes, about 20 minutes, about 30 minutes, about 45 minutes, about one hour, about one and a half hours, about 2 hours, about 3 hours, about 4 hours, about 6 hours, about 8 hours, about 12 hours, or about 24 hours, or more. In another embodiment, an elapsed time indicator indicates an elapsed time of at least 1 minute, at least 5 minutes, at least 10 minutes, at least 15 minutes, at least 20 minutes, at least 30 minutes, at least 45 minutes, at least one hour, at least one and a half hours, at least 2 hours, at least 3 hours, at least 4 hours, at least 6 hours, at least 8 hours, at least 12 hours, or at least 24 hours, or more.

In one embodiment, an elapsed time indicator produces a signal at regular intervals of the total elapsed time to be measured. For example, if the elapsed time indicator is designed to measure and indicate a total elapsed time of one hour, the indicator can produce a signal to indicate the point at which 15 minutes, 30 minutes, 45 minutes, and one hour have elapsed. Exemplary regular intervals include 1 minute intervals, 2 minute intervals, 5 minute intervals, 10 minute intervals, 15 minute intervals, 20 minute intervals, 30 minute intervals, 45 minute intervals, 60 minute intervals, 90 minute intervals, 120 minute intervals or more. Exemplary numbers of regular intervals within any total elapsed time period include 1, 2, 3, 4, 5, 6, 7, 8, 9, 10 or more intervals.

In another embodiment, an elapsed time indicator produces a continuous signal indicating the amount of time that has elapsed or that has yet to elapse (i.e., the time remaining). For example, an elapsed time indicator that is designed to measure and indicate a total elapsed time of one hour may be in the form of a progress bar, pie chart, or clock, wherein a signal is produced upon activation and increases linearly compared to the fraction of total elapsed time that has elapsed. In one embodiment, once the total time has elapsed the elapsed time indicator can discontinue producing the signal or produce a different signal to indicate that the total time has elapsed.

In a particular illustrative embodiment, the elapsed time indicating device comprises one or a plurality of any combination of elapsed time indicators. Each elapsed time indicator can be independently activated by a user or activated by exposure to a different predetermined temperature. In addition, each elapsed time indicator can measure and produce a signal for different predetermined lengths of time.

Merely for purposes of illustration, a single elapsed time indicating device may comprise: an elapsed time indicator that is user-activated and measures and indicates an elapsed time of 1 hour; an elapsed time indicator that is user-activated and measures and indicates an elapsed time of 2 hours; an elapsed time indicator that is user-activated and measures and indicates an elapsed time of 10 minutes; an elapsed time indicator that is activated by exposure to a predetermined temperature of 4° C. and measures and indicates an elapsed time of 1 hour, 2 hours, or more since activation; and/or an elapsed time indicator that is activated by exposure to a predetermined temperature in the range of 30° C.-40° C., and measures and indicates an elapsed time of 1 hours, 2 hours, or more since activation.

The invention contemplates that an elapsed time indicating device can comprise any number of elapsed time indicators that can be independently activated and that can independently measure and indicate when various predetermined lengths of time have elapsed.

Exemplary types of signals produced by elapsed time indicators include, but are not limited to visual signals, audible signals, infrared signals, radio signals, digital signals, analog signals, and combinations thereof.

In various illustrative embodiments, the elapsed time indicating device comprises an elapsed time indicator. A particular embodiment of the elapsed time indicator comprises (1) a reservoir containing a composition comprising a first reagent and/or a detectable agent, (2) a migration medium comprising a second reagent, through which the composition migrates in a repeatable and precise fashion, and (3) a barrier that separates the reservoir and the migration medium. The migration rate of the composition is linearly correlated to the amount of elapsed time. The rate of migration, i.e., elapsed time, can be calibrated, for example, by varying the viscosity of the composition, the molecular weight of the detectable reagent and/or the porosity of the migration medium.

Exemplary migration media are generally colorless or white, and include, without limitation, polymeric micro-porous materials, silica fillers, cellulose-based materials, sol-gels, and particle-filled Teflon® and combinations thereof.

In particular embodiments, the elapsed time indicator comprises a first and second reagent system and produces visual signal, e.g., a color change. Illustratively, the composition in the reservoir comprises a first reagent and/or a detectable agent and the migration medium comprises a second reagent which reacts with said first reagent, thereby allowing the first reagent to produce a color change by interacting or reacting with the detectable agent. The composition may further comprise, for example, water, a thixotropic material, an alcohol, or non-flammable solvent, or other organic species. For instance, the composition may further comprise a surfactant, a fatty acid, or an aliphatic alcohol.

Exemplary first and second reagent systems include, without limitation, chelation of a metal ion by a chelating agent, reaction of an acid/base with a pH indicator, reaction of an electron donor/acceptor with a redox indicator, and the enzymatic reaction between an enzyme and a substrate.

Exemplary metal ions include, without limitation, zinc ions, copper ions, iron ions and calcium ions and corresponding chelating agents include, without limitation, 1,10 phenanthroline, zincon, and 2,2′-biquioline (cupron) and 4-(2-pyridylazo)resorcinol monosodium salt (PAR). Exemplary acid/base reagents include, without limitation, hydrochloric acid, citric acid, ascorbic acid, sodium hydroxide and sodium hydrogen phosphate and corresponding pH indicators include, without limitation, bromothymol blue, methyl red and cresol red. Exemplary electron donor/acceptors include, without limitation, bleaching powder and vanadium salts and corresponding redox indicators include, without limitation, n-phenylanthrancilic acid and bleachable dyes.

In various illustrative embodiments, the elapsed time indicator comprises (1) a reservoir containing a composition comprising a dye, (2) a migration medium through which the composition migrates in a repeatable and precise fashion, and (3) a barrier that separates the reservoir and the migration medium. The migration rate of the composition is linearly correlated to the amount of elapsed time. The rate of migration, i.e., elapsed time, can be calibrated, for example, by varying the viscosity of the composition, the molecular weight of the dye and the porosity of the migration medium. In particular embodiments, the dyes do not require a chemical reaction to produce a visual signal, but instead the signal is progressively produced as a function of the migration rate of the colored dye through the uncolored or differently colored migration medium.

As will be clear to one skilled in the art, various types of graphic design may be employed for visualizing the elapsed time including, but not limited to, various lines, curves, ellipses, rectangles or points. Similarly, indicia showing the progress of the liquid-migration front may also be employed, including but not limited to various arrows, curves, lines and points of different sizes. For example, the signal can be viewed in a continuous window as progress on a progress bar or in a plurality of windows visible at particular intervals of elapsed time. Furthermore, each embodiment may be adapted to include numerous additional indicia to show the status of the time indicator. Such indicia include graphic symbols showing the gradual advance to the total elapsed time.

In one illustrative embodiment, a plurality of user-activated elapsed time indicators are present in the device, wherein each indicator can measure and indicate the same or different predetermined lengths of time. For example, user-activated elapsed time indicators can be independently activated by removing or disrupting a barrier, between the reservoir comprising the composition and the migration medium, which normally prevents migration of the composition through the membrane.

In another illustrative embodiment, a plurality of elapsed time indicators activated by exposure to a predetermined temperature or temperature range are present in the device, wherein each indicator can be activated by exposure to a different temperature and wherein the indicators can measure and indicate the same or different predetermined lengths of time.

For example, compositions comprising a detectable reagent and/or dye, as described herein or as known in the art, further comprise one or more compounds, wherein the fluidity of the compounds is altered by exposure to a particular temperature or temperature range. In this way, individual elapsed time indicators can be calibrated to become activated at different temperatures by selecting appropriate temperature dependent compounds. The rate of migration for each individual elapsed time indicator at each temperature can further be controlled by calibrating the particular molecular weight of the dye and pore size of the membrane.

In particular illustrative embodiments, suitable compounds include, for example, viscoelastic and viscous fluids and combinations thereof that provide the desired properties for migration in response to time and/or cumulative thermal exposure. Materials that exhibit both elastic and viscous properties simultaneously are called viscoelastic materials. A viscoelastic liquid can be identified as a viscoelastic material that continues to deform indefinitely when subjected to a shearing stress. In one embodiment, a viscoelastic material may exhibit a transition from an immobile, glassy state to a viscoelastic liquid state at a temperature known as the glass transition temperature, T_{g}. It may also exhibit a transition from a partially crystalline state to an amorphous state at the temperature at which the crystalline material melts, Tₘ. Often, such a material will behave as a viscoelastic solid below Tₘ. See, e.g., John D. Ferry, Viscoelastic Properties of Polymers, (John Wiley & Sons, Inc. 1980).

An illustrative, but by no means exclusive, list of viscoelastic and viscous materials that may be suitable for use in the articles of the present invention includes natural rubber; butyl rubber; polybutadiene and its copolymers with acrylonitrile and styrene; polyalpha-olefins such as polyhexene, polyoctene, and copolymers of these and others; polyacrylates; polychloroprene; polydimethylsiloxane; silicone oils and gums; mineral oils; and block copolymers such as styrene-isoprene block copolymers; and mixtures of any of the above. Materials that undergo a melting or glass transition to change from solid to liquid behavior and which could be useful in the present invention include hydrocarbon waxes, elastomer/tackifier blends, etc.

The viscoelastic materials may, for example, comprise elastomers conventionally formulated as pressure sensitive adhesives. Exemplary elastomers include, but are not limited to, polyisoprene, atactic polypropylene, polybutadiene, polyisobutylene, silicone, ethylene vinyl acetate, and acrylate based elastomers and can typically include a tackifying agent and/or a plasticizing agent.

Illustrative examples of related elapsed time indicating devices are described, for example, in U.S. Pat. Nos. 7,280,441; 7,232,253; 7,004,621; 6,752,430; 6,544,925; 6,042,264; 5,785,354; 5,719,828; 5,709,472; 5,667,303; 5,602,804; 5,368,905; 5,058,088; and 5,057,434.

In one illustrative embodiment, an elapsed time indicating device comprises a digital elapsed time indicator. In some embodiments, a digital elapsed time indicator is advantageous because it is easy to manufacture, is very thin and flexible, uses very small amounts of current, provides a clear visual display, and is easily calibrated at point-of-manufacture to a wide range of predetermined times. Moreover, digital elapsed time indicators can be integrated with a temperature sensor that activates the elapsed time indicator once the predetermined temperature has been reached.

The digital time elapse indicator includes a means of producing a signal that represents the passage of time such that a user will be informed not only of the total elapsed time but also of regular intervals of this time so as to gain an awareness of what fraction of the predetermined time period has elapsed and what fraction of the predetermined time period has yet to elapse. The signal is preferably a visual signal, more preferably an alphanumeric signal produced on an LED, OLED, or LCD.

In one embodiment, the digital elapsed time indicators comprise a short-range wireless interface, such as a BLUETOOTH interface, ZIGBEE, or WIFI interface, a long range cellular phone or satellite communications interface, or a wired interface, such as a RS 232, USB or FIREWIRE interface.

Illustrative examples of related digital elapsed time indicating device are described, for example, in U.S. Pat. Nos. 7,532,106; 7,362,663 and 5,802,015; and U.S. Patent Application Publication No.: 2009/0002185.

Environmental Condition Indicating Devices

The invention further contemplates, in part, vessels comprising an indicating device comprising one or more environmental indicators that measure, monitor, and indicate, for example, the pH, carbon dioxide concentration, oxygen concentration, osmolarity, or glucose concentration of the vessel's contents. In one embodiment, an environmental indicator continuously monitors and produces a signal that indicates the environmental condition. In another embodiment, an environmental indicators monitors and produces a signal at one or more predetermined times and/or at regular intervals. The signal is preferably a visual signal, more preferably an alphanumeric signal produced on an LED, OLED, or LCD.

In various embodiments, environmental indicators comprise digital sensors that measure, monitor, and indicate the environmental conditions in the vessel. In addition, it is contemplated that the sensors can be calibrated to measure any range of environmental conditions, without limitation. Thus, a normal range for each environmental condition can be pre-programmed into each environmental indicating device. The range can include one or more of a minimum threshold, optimal condition, or maximum threshold. In a particular embodiment, when the environmental condition being measured or monitored is outside of the minimum or maximum threshold values, the environmental indicator will produce an audible signal to indicate that the environmental condition is not within an acceptable range.

Other exemplary environmental indicators include, but are not limited to, Ca⁺⁺, potassium, sodium, magnesium, manganese, sulfate, phosphate, and chloride concentration.

Combination Indicating Devices

The invention, contemplates, in part, vessels comprising a combination of indicating devices that indicate, for example, the temperature of the vessel contents, the time the vessel has been at any given temperature, and optionally, one or more various environmental conditions (e.g., pH, oxygen concentration, glucose concentration). In preferred embodiments, the combination devices can include and of the features of individual devices and indicators disclosed herein. One having skill in the art would appreciate that the environmental indicating devices are preferably in contact with the contents of the vessel. The contemplated devices can be integrated into the material of the vessel at a point where the permeability of the vessel is such that the particular environmental sensing device is in contact with the vessel lumen or contents of the vessel. In another embodiment, the environmental indicating devices can be manufactured separately from the vessel and subsequently, permanently or non-permanently affixed or adhered to the outer surface of the vessel. In one related embodiment, the affixed or adhered device perforates the vessel such that the particular environmental sensing device is in contacts with the vessel lumen or contents of the vessel. In another related embodiment, the affixed or adhered device is applied to a portion of the device that is permeable, such that the particular environmental sensing device is in contact with the vessel lumen or contents of the vessel.

In a preferred embodiment, the vessel comprises a combination of a temperature indicating device and an elapsed time indicating device, either separately as individual devices or as a single device. In further embodiments, the vessel comprises a combination of a temperature indicating device, an elapsed time indicating device and one or more environmental condition indicating devices either separately as individual devices or as a single device.

The combination indicating device can be fabricated integral with the vessel, can be permanently or non-permanently attached to the vessel exterior surface, can be laminated to the vessel exterior surface or can be encased with the vessel within a vessel liner. The device can be a small electronic device, a probe, a sensor, or a combination thereof.

The combination indicating device may be of any size or shape.

Exemplary shapes of the combination indicating device, include, but are not limited to: cards, strips, disks, stickers, labels, probes, or combinations thereof. For example a temperature indicating device in the form of a card or strip may comprise one or a plurality of temperature indicating disks or vice versa.

As will be clear to one skilled in the art, various types of graphic design may be employed for visualizing the elapsed time and temperature of the vessel including, but not limited to, various lines, curves, ellipses, rectangles or points. Similarly, indicia showing the progress of the liquid-migration front may also be employed, including but not limited to various arrows, curves, lines and points of different sizes. For example, the signal can be viewed in a continuous window as progress on a progress bar or in a plurality of windows visible at particular intervals of elapsed time for particular temperatures or a range of temperatures. Furthermore, each embodiment may be adapted to include numerous additional indicia to show the status of the time indicator or temperature of the vessel. Such indicia include graphic symbols showing the gradual advance to the total elapsed time versus temperature.

In a more preferred embodiment, the vessel comprises a combination of a temperature indicating device and an elapsed time indicating device. In other embodiments, the signals from any of the foregoing devices on a vessel of the invention is transmitted to and displayed on a closed cell processing system used in combination with the vessel.

### C. Cells

In various embodiments, a vessel of the invention comprises a population of cells. The cells may be any type of cells, including bacteria, yeast, plant cells, mammalian cells, human cells, cell lines, primary cells, embryonic or adult stem cells, or induced pluripotent stem cells. The cells may be derived from or contained within bodily fluids, such as blood, urine, saliva, or cerebrospinal fluid, and in particular embodiments the source of the cells is human umbilical cord blood, human bone marrow, or human mobilized peripheral blood. In some embodiments, the cells may be used in ex-vivo or in vitro cell therapy. The source of the cells can be allogenic, syngenic, autogenic or xenogenic in nature. The source of the cells is preferably mammalian and more preferably, human.

In one embodiment, a population of cells is selected from adult or neonatal stem/progenitor cells, including mesodermal stem/progenitor cells, endodermal stem/progenitor cells, and ectodermal stem/progenitor cells.

Illustrative examples of mesodermal stem/progenitor cells include, but are not limited to mesenchymal stem/progenitor cells, endothelial stem/progenitor cells, bone marrow stem/progenitor cells, umbilical cord stem/progenitor cells, adipose tissue derived stem/progenitor cells, hematopoietic stem/progenitor cells (HSGs), umbilical cord stem/progenitor cells, placental stem/progenitor cells, stem/progenitor cells obtained from the peripheral blood, muscle stem/progenitor cells, kidney stem/progenitor cells, osteoblast stem/progenitor cells, chondrocyte stem/progenitor cells, and the like.

Illustrative examples of ectodermal stem/progenitor cells include, but are not limited to neural stem/progenitor cells, retinal stem/progenitor cells, skin stem/progenitor cells, and the like.

Illustrative examples of endodermal stem/progenitor cells include, but are not limited to liver stem/progenitor cells, pancreatic stem/progenitor cells, epithelial stem/progenitor cells, and the like.

Other illustrative examples of population of cells suitable for use in the invention include isolated heterogeneous or homogeneous populations of cells selected from the group consisting of: pancreatic islet cells, CNS cells, PNS cells, cardiac muscle cells, skeletal muscle cells, smooth muscle cells, hematopoietic cells, bone cells, liver cells, an adipose cells, renal cells, lung cells, chondrocyte, skin cells, follicular cells, vascular cells, epithelial cells, immune cells, endothelial cells, and the like.

In other illustrative embodiments, the population of cells comprises induced pluripotent stem cells.

### D. Cell Culture Media

Cell populations of the invention may be exposed to a plurality of conditions, including culture conditions, treatment conditions, storage conditions, conditions suitable for manipulation and handling, and conditions suitable for administration to an individual.

In various embodiments, vessels of the invention comprise a population of cells and any suitable cell culture medium. Suitable cell culture media include, but are not limited to, cell culture growth media, conditioned cell culture media, serum-free media, cell-freezing media, and pharmaceutically acceptable cell culture media, the formulations of which are disclosed in the art.

In some embodiments, it may be desirable to replace the cell culture media at regular or predetermined intervals with the same or different cell culture medium and/or a physiologically acceptable buffer (e.g., PBS, Ringer's solution, physiological saline solution).

For example, a population of cells may be grown in a cell culture growth medium; treated in a cell culture medium comprising one or more pharmaceutical agents; and either frozen or stored in a freezing medium or administered to an individual in a pharmaceutically acceptable cell culture medium. A population of cells may also require exchange between cell culture media depending on the application, and additionally, may require or benefit from a “wash” step, wherein the exchange of cell culture media involves an intermediate step of exposing the cells to phosphate buffered saline, or another suitable physiological buffer (e.g., saline, Ringer's). Exchange of the media may be accomplished via one or more ports in the vessel.

### E. Pharmaceutical Agents

In various illustrative embodiments, the invention contemplates, in part, a vessel comprising a population of cells, a cell culture medium, and a pharmaceutical agent. In particular embodiments, a population of cells is contacted in the vessel with one or more pharmaceutical agents in an amount and for a duration sufficient to treat the cells. In some embodiments, the cells are treated with a pharmaceutical agent to increase the proliferation, engraftment or homing potential of the cells relative to a population of cells that has not been contacted with the one or more pharmaceutical agents. The cells may be contacted in the vessel with one or more pharmaceutical agents sequentially or at the same time in a single composition. Alternatively, the population of cells may be contacted with a plurality of compositions, each comprising one or more pharmaceutical agents, individually or at the same time. In addition, the population of cells may be contacted with a plurality of compositions, wherein the medium is exchanged and/or the cells are washed between each treatment. The increased cell proliferation may occur in the vessel during an extended treatment or may occur once the cells have been administered to an individual after a treatment of brief duration. Pharmaceutical agents and compositions are preferably endotoxin-free and stored in sterile vessels prior to contacting the population of cells.

Exemplary treatments of an extended duration generally include a treatment time of more than 12 hours, more than 18 hours, more than 24 hours, more than 36 hours or more. In a particular embodiment, a treatment of an extended duration is the time it takes for one or more population doublings to occur.

Exemplary treatments of a brief duration generally include a treatment time of less than 12 hours, less than 6 hours, less than 5 hours, less than 4 hours, less than 3 hours, less than 2 hours, less than 1 hour or less. In other embodiments, treatments of a brief duration generally include treatment times of about 5 minutes, about 10 minutes, about 15 minutes, about 20 minutes, about 30 minutes, about 45 minutes, about 1 hour, about 2 hours, about 3 hours, about 4 hours, about 5 hours, or about 6 hours. In a particular embodiment, a treatment of brief duration is any amount of time before a first population doubling occurs.

In various embodiments, the populations of cells undergo a plurality of treatments with one or a plurality of compositions, each comprising one or more pharmaceutical agents. The populations of cells may be contacted with the same or with different compositions for the same or for different periods of time. An advantage of the presently contemplated vessels is that such manipulations may be accomplished in the same vessels through the input and output ports, wherein the medium is exchanged and/or the cells are washed between each treatment without any difficulty.

The contemplated vessels have devices for indicating temperature and/or environmental conditions of the vessels, allowing precise control over incubation conditions for incubating a population of cells with an agent, including incubation temperature and time.

In particular embodiments, a population of cells is treated (e.g., contacted) with one or more pharmaceutical agents, each at a final concentration of about 1 μM to about 100 μM. In certain embodiments, a population of cells is treated with one or more pharmaceutical agents, each at a final concentration of about 1×10⁻¹⁴ M to about 1×10⁻³ M, about 1×10⁻¹³ M to about 1×10⁻⁴ M, about 1×10⁻¹² M to about 1×10⁻⁵ M, about 1×10⁻¹¹ M to about 1×10⁻⁴ M, about 1×10⁻¹¹ M to about 1×10⁻⁵ M, about 1×10⁻¹⁰ M to about 1×10⁻⁴ M, about 1×10⁻¹⁰ M to about 1×10⁻⁵ M, about 1×10⁻⁹ M to about 1×10⁻⁴ M, about 1×10⁻⁹ M to about 1×10⁻⁵ M, about 1×10⁻⁸ M to about 1×10⁻⁴ M, about 1×10⁻⁷ M to about 1×10⁻⁴ M, about 1×10⁻⁶ M to about 1×10⁻⁴ M, or any intervening ranges of final concentrations.

In another particular embodiment, a population of cells is treated with one or more pharmaceutical agents, each at a final concentration of about 1×10⁻¹⁴ M, about 1×10¹³ M, about 1×10⁻¹² M, about 1×10⁻¹⁰ M, about 1×10⁻⁹ M, about 1×10⁻⁸ M, about 1×10⁻⁷ M to about 1×10⁻⁶ M, about 1×10⁻⁵ M, about 1×10⁻⁴ M, about 1×10⁻³ M, or any intervening final concentration. In treatments comprising one or more pharmaceutical agents, the agonists can be at different concentrations from each other or at the same concentration.

In particular embodiments, a population of cells is treated (e.g., contacted with one or more pharmaceutical agents) 1, 2, 3, 4, 5, 6, 7, 8, 9 or 10 or more times. A population of cells can be intermittently, episodically, or sequentially contacted with one or more pharmaceutical agents within the same vessel (e.g., contacting the population of cells with one drug for a period of time, exchanging the culture medium and/or washing the population of cells, then repeating the cycle with the same or a different combination of pharmaceutical agents for the same predetermined period of time or a different predetermined period of time).

Pharmaceutical compositions of the invention may comprise one or more pharmaceutical agents as described herein, formulated in pharmaceutically-acceptable or physiologically-acceptable solutions for administration to a vessel comprising a population of cells. It will also be understood that, if desired, the compositions of the invention may be administered in combination with other agents as well, such as, e.g., other proteins or polypeptides or various pharmaceutically-active agents. There is virtually no limit to other components that may also be included in the compositions, provided that the additional agents do not adversely affect the proliferative effects desired to be achieved.

In the pharmaceutical compositions of the invention, formulation of pharmaceutically-acceptable excipients and carrier solutions is well-known to those of skill in the art. As such, these compositions may be formulated with an inert diluent, for example, water, ethanol, or a polyol (e.g., glycerol, propylene glycol, and liquid polyethylene glycol, and the like). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

Exemplary pharmaceutical agents include, but are not limited to, a parathyroid hormone/parathyroid related peptide (PTH/PTHrP) polypeptides, cAMP enhancers, growth factors, cytokines, a prostaglandin receptor 2 agonist (PGE-2), non-PGE2 prostaglandin agonists, a Wnt pathway agonist, and a Notch pathway agonist, and active fragments and derivatives thereof.

Illustrative examples of PTH/PTHrP polypeptides include, but are not limited to, PTH, PTHrP, PTH(1-84), PTH(1-31), PTH(1-34), PTH(1-36), PTH(1-37), PTH(1-38), PTH(1-41), PTH(28-48) and PTH(25-39), stability-enhanced variants of PTH are known in the art from e.g. WO 92/11286 and WO 93/20203, and cyclized PTH analogues are disclosed in e.g. WO 98/05683.

Illustrative examples of cAMP enhancers include, but are not limited to, dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, prostaglandin E1, prostaglandin E2, pituitary adenylate cyclase activating polypeptide (PACAP), and vasoactive intestinal polypeptide (VIP), and active fragments and derivatives thereof.

In particular embodiments, the EP receptor agonist preferentially interacts with, and is selective for, PGE2 EP4 or EP2 receptors. An agonist is selective for the PGE2 EP4 receptor if the agonist has a higher binding affinity for the PGE2 EP4 receptor as compared to its binding affinity for the PGE2

EP1, EP2, and EP3 receptors. Illustrative examples of prostaglandin EP receptor agonists include, but are not limited to, prostaglandin E2 (PGE2), 16,16-dimethyl PGE2, 16-16 dimethyl PGE2 p-(p-acetamidobenzamido) phenyl ester, 11-deoxy-16,16-dimethyl PGE2, 9-deoxy-9-methylene-16,16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15(R)-15-methyl PGE2, 8-iso-15-keto PGE2, 8-iso PGE2 isopropyl ester, 20-hydroxy PGE2, 11-deoxy PGE1, nocloprost, sulprostone, butaprost, 15-keto PGE2, and 19 (R) hydroxy PGE2, and active fragments and derivatives thereof.

In particular embodiments, a population of cells is treated with one or more prostaglandin EP receptor agonists, each at a final concentration of about 1×10¹³ M to about 1×10⁻⁴ M, about 1×10⁻¹² M to about 1×10⁻⁴ M, about 1×10¹¹ M to about 1×10⁻⁴ M, about 1×10⁻¹⁰ M to about 1×10⁻⁴ M, about 1×10⁻⁹ M to about 1×10⁻⁴ M, about 1×10⁻⁵ M to about 1×10⁻⁴ M, about 1×10⁻⁷ M to about 1×10⁻⁴ M, about 1×10⁻⁶ M to about 1×10⁻⁴ M, or any intervening ranges of final concentrations. In some embodiments, the population of cells is treated with an EP4 specific agonist, such as 16,16-dimethyl PGE2, at a final concentration of about 1×10⁻¹³ M to about 1×10⁻⁴ M, about 1×10⁻¹² M to about 1×10⁻⁴ M, about 1×10⁻¹¹ M to about 1×10⁻⁴ M, about 1×10⁻¹⁰ M to about 1×10⁻⁴ M, about 1×10⁻⁹ M to about 1×10⁻⁴ M, about 1×10⁻⁵ M to about 1×10⁻⁴ M, about 1×10⁻⁷ M to about 1×10⁻⁴ M, about 1×10⁻⁶ M to about 1×10⁻⁴ M, or any intervening ranges of final concentrations.

In another particular embodiment, a population of cells is treated with one or more prostaglandin EP receptor agonists, each at a final concentration of about 1×10⁻¹⁴ M, about 1×10⁻¹³ M, about 1×10⁻¹² M, about 1×10⁻¹⁰ M, about 1×10⁻⁹ M, about 1×10⁻⁵ M, about 1×10⁻⁷ M to about 1×10⁻⁶ M, about 1×10⁻⁵ M, about 1×10⁻⁴ M, about 1×10⁻³ M, or any intervening final concentration. In some embodiments, the population of cells is treated with an EP4 specific agonist, such as 16,16-dimethyl PGE2, at a final concentration of about 1×10⁻¹⁴ M, about 1×10⁻¹³ M, about 1×10⁻¹² M, about 1×10⁻¹⁰ M, about 1×10⁻⁹ M, about 1×10⁻⁵ M, about 1×10⁻⁷ M to about 1×10⁻⁶ M, about 1×10⁻⁵ M, about 1×10⁻⁴ M, about 1×10⁻³ M, or any intervening final concentration.

In preferred embodiments, a population of cells is treated with one or more prostaglandin EP receptor agonists, each at a final concentration of about 1 μM to about 100 μM. In certain preferred embodiments, a population of cells is treated with one or more prostaglandin EP receptor agonists, each at a final concentration of 1 μM, 10 μM, or 100 μM. In treatments comprising one or more agonists, the agonists can be at different concentrations from each other or at the same concentration. In some embodiments, the population of cells is treated with an EP4 specific agonist, such as 16,16-dimethyl PGE2, at a final concentration of about 1 μM to about 100 μM, or at a final concentration of 1 μM, 10 μM, or 100 μM.

Illustrative examples of non-PGE2 prostaglandin agonists include, but are not limited to, an EP1 agonist, e.g., ONO-DI-004 and ONO-8713; an EP2 agonist, e.g., CAY10399, ONO_{—}8815Ly, ONO-AE1-259, and CP-533,53;

an EP3 agonist, e.g., AE5-599, MB28767, GR 63799X, ONO-NT012, and ONO-AE-248; and an EP4 agonist, e.g., ONO-4819, APS-999 Na, AH23848, and ONO-AE1-329, and active derivatives thereof.

Illustrative examples of growth factor or cytokines include, but are not limited to, brain derived neurotrophic factor (BDNF), bone morphogenetic protein 2 (BMP-2), bone morphogenetic protein 6 (BMP-6), bone morphogenetic protein 7 (BMP-7), cardiotrophin 1 (BMP-2), CD22, CD40, ciliary neurotrophic factor (CNTF), CCL1-CCL28, CXCL1-CXCL17, XCL1, XCL2, CX3CL1, vascular endothelial cell growth factor (VEGF), epidermal growth factor (EGF), FAS-ligand, fibroblast growth factor 1 (FGF-1), fibroblast growth factor 2 (FGF-2), fibroblast growth factor 4 (FGF-4), fibroblast growth factor 5 (FGF-5), fibroblast growth factor 6 (FGF-6), fibroblast growth factor 1 (FGF-7), fibroblast growth factor 1 (FGF-10), Flt-3, granulocyte colony-stimulating factor (G-CSF), granulocyte macrophage stimulating factor (GM-CSF), hepatocyte growth factor (HGF), interferon alpha (IFN-a), interferon beta (IFN-b), interferon gamma (IFNg), insulin-like growth factor 1(IGF-1), insulin-like growth factor 2 (IGF-2), interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 15 (IL-15), interleukin 17

(IL-17), interleukin 19 (IL-19), macrophage colony-stimulating factor (M-CSF), monocyte chemotactic protein 1 (MCP-1), macrophage inflammatory protein 3a (MIP-3a), macrophage inflammatory protein 3b (MIP-3b), nerve growth factor (NGF), neurotrophin 3 (NT-3), neurotrophin 4 (NT-4), platelet derived growth factor AA (PDGF-AA), platelet derived growth factor AB (PDGF-AB), platelet derived growth factor BB (PDGF-BB), platelet derived growth factor CC (PDGF-CC), platelet derived growth factor DD (PDGF-DD), RANTES, stem cell factor (SCF), stromal cell derived factor 1 (SDF-1), transforming growth factor alpha (TGF-a), transforming growth factor beta (TGF-b), and tumor necrosis factor alpha (TNF-a), and active fragments and derivatives thereof.

Illustrative examples of Wnt pathway agonists include, but are not limited to, a GSK-3β inhibitor, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt10a, Wnt10b, Wnt11, Wnt14, Wnt15, and Wnt16, and active fragments and derivatives thereof.

Illustrative examples of Hedgehog pathway agonists include, but are not limited to, retinoic acid, Sonic hedgehog, Desert hedgehog, and Indian hedgehog, and active fragments and derivatives thereof.

Illustrative examples of Notch pathway agonists include, but are not limited to, Notch-1, Notch-2, Notch-3, Notch-4, Jagged 1, Jagged 2, Lunatic-Fringe, Manic-Fringe, Radical Fringe, Delta, Serrate, and active fragments and derivatives thereof.

### F. Kits

The present invention also contemplates, in part, a kit comprising an endotoxin free vessel as described elsewhere herein, a population of cells as described elsewhere herein, one or more pharmaceutical agents as described elsewhere herein, and instructions for using the kit.

In particular illustrative embodiments, the kit comprises an endotoxin free vessel comprising at least one temperature indicating device. In a preferred embodiment, the vessel comprises at least one temperature indicating device and at least one elapsed time indicating device as described elsewhere herein. In related embodiments, the vessel comprises at least one temperature indicating device, at least one elapsed time indicating device, and at least one environmental condition indicating device.

In one embodiment, the kit comprises a population of hematopoietic stem cells and 16,16 dimethyl PGE2 supplied in dimethyl sulfoxide (DMSO). Preferably, the 16,16 dimethyl PGE2 in DMSO is supplied in a 2 ml vial with a Teflon coated stopper, wherein there is an air overlay in the vial.

In particular embodiments, kits comprise PGE2 in DMSO at a concentration of about 1×10⁻⁴ M to about 1 M, about 1×10⁻³ M to about 1 M, about 1×10⁻² M to about 1 M, about 1×10⁻³ M to about 1 M, about 1×10⁻⁴ M to about 1×10⁻² M, about 1×10⁻⁴ M to about 1×10⁻³ M, about 1×10⁻³ M to about 1×10⁻² M, or any intervening ranges of final concentrations.

In another particular embodiment, kits comprise PGE2 in DMSO at a concentration of about 1×10⁻⁴ M, about 1×10⁻³ M, about 1×10⁻² M, about 1 M, or any intervening final concentration.

In preferred embodiments, kits comprise PGE2 in DMSO at a concentration of about 10 mM to about 1 M. In certain preferred embodiments, a kit comprises PGE2 in DMSO at a concentration of 10 mM, 100 mM, or 1 M. In one embodiment, a kit comprises PGE2 in DMSO at a concentration of about 10 mM.

### G. Cell Therapy

The invention contemplates that a vessel as disclosed herein, is suitable for multiple continuous or discontinuous steps of culturing, handling, manipulating, storing, analyzing, incubating, administering and otherwise establishing, supporting, harvesting, and using cells and by-products thereof ex vivo or in vitro or otherwise. Cells in the vessel of the invention can be treated to aid in the growth or proliferation of cells, or alter the activity or characteristics of the cells. In some embodiments, a population of cells is treated in a vessel of the invention having temperature and elapsed time indicating devices to enhance cell survival, proliferation, engraftment, or homing.

A particular advantage of the contemplated vessels is the ability to use one vessel from the initial cell collection and cell culture steps to the step of administering the cells or by-products to a patient. In particular embodiments, once the cells have been collected or isolated from an individual and transferred to a vessel as described herein, the cells are not transferred to another vessel prior to administration to an individual. The vessels of the invention thus reduce handling and processing steps of the cells that may decrease cell viability.

In particular embodiments, vessels are used in combination with a closed cell processing system. In some embodiments, a vessel of the invention is a component in a closed cell processing system. Optionally, one or more handling steps such as treatment, centrifugation, incubation, media addition, cell selection, cell washing, and final fill and finish within “closed” or resealable vessels of the invention is automated, which may enhance the quality and reproducibility of cell culture manipulations.

In various embodiments, the vessels of the invention are used to prepare cells for ex vivo or in vitro cell therapy. In some embodiments, the cells are prepared for transplantation and other therapies, particularly for hematologic and oncologic diseases. Ex vivo and in vitro methods are useful because a significant number of autologous donor transplants contain insufficient populations of the desired cells. Likewise, individuals are often unable to find histocompatible donors, emphasizing the need for improved methods for expanding cell populations for transplantation.

In one embodiment, the invention provides a method of preparing a population cells to administer to an individual in need of cell therapy comprising: providing a population of cells to be cultured; culturing the population of cells in an endotoxin-free vessel as described elsewhere herein; contacting the population of cells with one or more pharmaceutical agents in the vessel as described elsewhere herein, under conditions sufficient to prepare the cells for administering to an individual; and administering the prepared population of cells to the individual.

In one preferred embodiment, the ex-vivo cell therapy methods contemplated herein, are carried out in one endotoxin-free vessel as described herein. In particular embodiments, the vessel is a closed cell processing system, is used in combination with a closed cell processing system, or is a component of a closed cell processing system.

Illustrative examples of individuals in need of cell therapy include, but are not limited to, a bone marrow donor who has donated bone marrow, a bone marrow donor who has yet to donate bone marrow, a bone marrow donor transplant recipient, an individual that has hematopoietic progenitor cells under environmental stress, an individual that has anemia, an individual that has a reduced level of immune cell function compared to a normal subject, or an individual that has an immune system deficiency.

Other illustrative examples of individuals in need of cell therapy include individuals with myeloma, non-Hodgkin's lymphoma, Hodgkin's lymphoma, chronic myeloid leukemia, chronic myelogenous leukemia, chronic granulocytic leukemia, acute lymphoblastic leukemia, acute nonlymphoblastic leukemia, or pre-leukemia.

Exemplary methods of administration include, parenteral administration selected from the group consisting of: intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In one embodiment, a population of hematopoietic cells is prepared in a vessel of the invention for administration to an individual in need thereof. Using a vessel of the invention, a cell population may be collected, precisely treated at a desired temperature and time using the temperature and time indicating devices, washed to remove drug reagent from the vessel, and administered from the same vessel. A population of cells comprising hematopoietic stem cells is obtained from the bone marrow, peripheral blood, umbilical cord blood, or placenta. The heterogeneous mixture of cells is collected in or transferred to a vessel comprising a temperature indicating device and an elapsed time indicating device as described elsewhere herein, and a suitable growth medium is provided. The population of cells is contacted in the vessel with a pharmaceutical composition comprising 16,16-dimethyl PGE₂ in DMSO at a final concentration of 10 μM of 16,16-dimethyl PGE₂. The cells are incubated in the vessel at an incubation temperature of about 4° C. (e.g., in an ice bath), or alternatively, at about room temperature (about 22° C.) or about body temperature (about 37° C.), for an incubation period of about ten minutes to about one hour, preferably about one hour. A temperature indicator provides a signal to indicate the temperature of the vessel contents; an elapsed time indicator is activated when the vessel contents are exposed to the desired incubation temperature (i.e., about 4° C., 22° C. or 37° C.), and indicates when the incubation period (i.e., about ten minutes, about 15 minutes, about one hour) at the incubation temperature has elapsed. After incubation, the cell culture medium is exchanged and the vessel comprising the cells is warmed to body temperature. Once the proper temperature has been reached, a temperature indicator on the vessel produces a signal that the cells are ready to be administered to the individual.

Use of a vessel of the invention having temperature indicators and elapsed time indicators as disclosed herein to indicate exposure for a predetermined period of time at the desired temperature or temperature range allows precise control of the treatment time and temperature. Treating cells in a single vessel, and then washing cells in the vessel to remove drug substance from the vessel, also diminishes harmful extra steps in handling and processing of the cells, and additionally provides an assurance that the clinical protocol for treating cells has been properly carried out at the proper temperature and for the proper time period.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An endotoxin-free vessel comprising:
a) a temperature indicating device comprising at least one temperature indicator that produces a signal that indicates the temperature of the vessel;
b) an elapsed time indicating device that comprises at least one elapsed time indicator; and
c) a population of cells;
wherein the vessel is suitable for cell culture, cell storage, cell infusion, treatment of cells, or cell therapy.

2. The vessel of claim 1, wherein the vessel comprises at least one input port and at least one output port.

3. (canceled)

4. The vessel of claim 1, wherein the at least one temperature indicator;
(a) is reversible;
(b) produces a signal that indicates that the temperature of the vessel is above, below, or within a target temperature range
(c) produces a signal that comprises a color that corresponds to the temperature of the vessel that is above, below. or within a target temperature range;
(d) produces a signal that is a digital indication of the temperature of the vessel:
(e) comprises a temperature scale that indicates a temperature of the vessel, wherein the temperature indicated is in the range 0° C. to 65° C.:
(f) comprises a temperature scale that indicates a temperature of the vessel. wherein the temperature indicated is in the range 0° C. to 50° C.; or
(g) comprises a temperature scale that indicates a temperature of the vessel. wherein the temperature indicated is in the range 4° C. to 37° C.

5. (canceled)

6. (canceled)

7. (canceled)

8. (canceled)

9. (canceled)

10. (canceled)

11. The vessel of claim 1, wherein the at least one elapsed time indicator
(a) produces a signal that indicates the elapsed time; or
(b) is activated by a user or by the temperature of the vessel reaching a predetermined threshold temperature.

12. (canceled)

13. The vessel of claim 11, wherein the elapsed time signal;
(a) indicates that the vessel has been exposed to a predetermined temperature range for a predetermined elapsed time
(b) indicates the elapsed time is selected from the group consisting of: a visual signal, an audible signal, an infrared signal, a radio signal, a digital signal, and an analog signal;
(c) is indicated at predetermined intervals; or
(d) is indicated continuously.

14. The vessel of claim 11, wherein:
(a) the elapsed time is at least 10 minutes;
(b) the elapsed time is at least 1 hour:
(c) the elapsed time is at least 2 hours; or
(d) the elapsed time is at least 4 hours.

15. (canceled)

16. (canceled)

17. (canceled)

18. (canceled)

19. (canceled)

20. (canceled)

21. The vessel of claim 1, comprising an environmental condition indicating device comprising at least one environmental condition indicator that indicates an environmental condition in the vessel selected from the group consisting of: pH, oxygen concentration, carbon dioxide concentration, osmolarity, and glucose concentration.

22. (canceled)

23. The vessel of claim 1, wherein the vessel is selected from the group consisting of: a bag, a pouch, a flask, a three dimensional matrix, and a bioreactor.

24. (canceled)

25. (canceled)

26. (canceled)

27. (canceled)

28. The vessel of claim 1, wherein the population of cells is selected from the group consisting of: stem cells, induced pluripotent stem cells, and progenitor cells.

29. The vessel of claim 28, wherein the population of cells is obtained from peripheral blood, umbilical cord blood, bone marrow, or placenta.

30. The vessel of claim 28, wherein the population of cells comprises hematopoietic stem and/or progenitor cells.

31. The vessel of claim 1, comprising a cell culture medium.

32. The vessel of claim 31, wherein the cell culture medium:
(a) is a cell growth medium;
(b) is serum free;
(c) is replaced at regular intervals with another cell culture medium;
(d) is replaced with a cell storage medium;
(e) is replaced with a pharmaceutically acceptable cell culture medium.

33. (canceled)

34. (canceled)

35. (canceled)

36. (canceled)

37. The vessel of claim 1, comprising one or more pharmaceutical agents.

38. The vessel of claim 37, wherein the one or more pharmaceutical agents are selected from the group consisting of: a PTH/PTHrP polypeptide, a prostaglandin receptor 2 agonist, a selective agonist of the PGE2 EP4 receptor, a Wnt pathway agonist, a Hedgehog pathway agonist, and a Notch pathway agonist.

39. The vessel of claim 1, comprising a population of hematopoietic stem cells and a pharmaceutical agent, wherein the pharmaceutical agent comprises 16,16 dimethyl PGE2 supplied in dimethyl sulfoxide (DMSO).

40. The vessel of claim 39, wherein the DMSO is substantially free of methyl acetate, and wherein the 16,16 dimethyl PGE2 is used at a final concentration of about 1 μM to about 100 μM.

41. The vessel of claim 39, wherein the DMSO is substantially free of methyl acetate, and wherein the 16,16 dimethyl PGE2 is used at a final concentration of about 10 μM.

42. A vessel according to claim 1, wherein the vessel forms or is a component of a closed cell processing system.

43. The vessel of claim 42, wherein the closed cell processing system automates one or more cell processing steps, selected from the groups consisting of: cell incubation cell culture, cell treatment, cell storage, centrifugation, cell selection media addition media exchange cell washing and cell infusion.

44. (canceled)
